(19) United States
(12) Patent Application Publication
(10) Pub. No.: US 2009/0137632 A1
(43) Pub. Date: May 28, 2009

(54) Compositions And Methods For Treating Dyslipidemia

(76) Inventors: M. Johnston Erwin, Indianapolis, IN (US); Mohamed Wa'el Ahned Hashad, Carmel, IN (US); Mark Chandrakant Lakshmanan, Zionsville, IN (US); Iris Rajman, Hampshire (GB)

Correspondence Address: ELI LILLY & COMPANY, PATENT DIVISION, P.O. BOX 6288, INDIANAPOLIS, IN, 46206-6288, US

(21) Appl. No.: 11/813,909

(22) PCT Filed: Feb. 2, 2006
(86) PCT No.: PCT/US06/03589

§ 371 Date: Jul. 13, 2007

Related U.S. Application Data
(60) Provisional application No. 60/652,238, filed on Feb. 11, 2005.
(60) Provisional application No. 60/655,845, filed on Feb. 24, 2005.
(60) Provisional application No. 60/664,082, filed on Mar. 22, 2005.

(51) Int. Cl. A61K 31/4535; A61P 9/10

(52) U.S. Cl. 514/324

(57) ABSTRACT

The present invention provides a method for treating dyslipidemia in a mammal, comprising administering to a mammal in need thereof, an effective amount of a compound of Formula (I): or a pharmaceutically acceptable salt or solvate thereof and at least one statin.

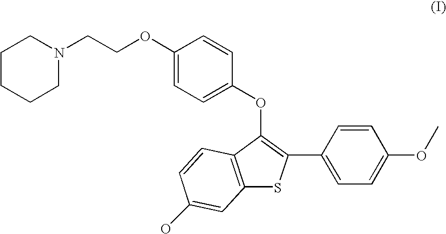

This application claims the benefit of U.S. Provisional Patent Application No. 60/664,082, filed Mar. 22, 2005.

### FIELD OF THE INVENTION

The present invention relates to the use of a combination of 2-(4-Methoxyphenyl)-4-[4-[2-(1-piperidinyl)ethoxy]phenyoxy]benzo[b]thiophene-6-ol and HMG CoA reductase inhibitors, (herein after referred to as “statins”) for the treatment of dyslipidemia. The compound 2-(4-Methoxyphenyl)-4-[4-[2-(1-piperidinyl)ethoxy]phenyoxy]benzo[b]thiophene-6-ol is also known as arzoxifene (hereinafter, with its pharmaceutically acceptable salts, solvates, and polymorphic forms collectively referred to as “arzoxifene”).

### BACKGROUND OF THE INVENTION

The compound, arzoxifene, employed in the method of the present invention is known. The compound, methods of preparing the compound, as well as pharmaceutical formulations containing the compound, are described in U.S. Pat. No. 5,723,474 (herein “'474 patent”). The '474 patent discloses that arzoxifene can be useful for the treatment of the various medical indications associated with post-menopausal syndrome, hyperlidemia, and aortal smooth muscle cell proliferation. Arzoxifene is known to be useful for treatment of osteoporosis. Polymorphic and solvate forms of arzoxifene are known in the art.

It has been reported that an additive effect is observed when the SERM compound, Raloxifene, is co-administered with a statin. Cheryl A. Keech, Year-by-year analysis of cardiovascular events in the Multiple Outcomes of Raloxifene Evaluation (MORE) trial, 21 CURRENT MEDICAL RESEARCH AND OPINION, No. 1, 135-140(2005).

One published US patent application, US2003/01/0162807, claims a method for promoting bone formation, and/or preventing bone loss and/or lowering blood cholesterol using an estrogen agonist/antagonist and a statin. Although Arzoxifene is stated to be a SERM, Applicants are aware of no reports of the use of Arzoxifene with a statin to synergistically treat dyslipidemia. More particularly, there are no reports describing the use of Arzoxifene and a statin for synergistic treatment of dyslipidemia in clinical patients in need of lipid profile improvement.

It has been established that lowering LDL-C levels affords protection from coronary heart disease (see, e.g., The Scandinavian Simvastatin Survival Study Group: Randomized trial of cholesterol lowering in 4444 patients with coronary heart disease: the Scandinavian Simvastatin Survival Study (4S), Lancet, 1994; 344:1383-89; and Shepherd, J. et al., Prevention of coronary heart disease with pravastatin in men with hypercholesterolemia, N Engl J Med, 1995; 333:1301-07). Additional treatment options are desired for patients suffering from or susceptible to dyslipidemia. Treatment options that can effectively treat dyslipidemia while providing beneficial therapeutic effects on certain indications associated with post-menopausal syndrome would be particularly desired. It is further desired to substantially reduce the concomitant liability of adverse effects associated with estrogen administration.

Thus, there is a need for development of new pharmaceutical treatments that can be used to treat dyslipidemia. Statins, currently used for treatment, at high doses may be associated with muscle pain due to myositis. This muscle pain is more frequently found in older women. R.S. Rosenson, Current overview of statin-induced myopathy, AM J Med., 2004; 116:408-416. An especially desired treatment is one that can offer a desirable side effect profile and a favorable effect on the patient's lipid profile that is greater than either arzoxifene alone or the statin alone. An Arzoxifene and statin combination can address the need for a treatment that can lower some lipids and ApoB more than either agent administered alone. Further, the co-administration of Arzoxifene and a statin can provide a favorable effect on HDL-C. The present invention can fulfill the patient's desire for a convenient, improved treatment for dyslipidemia and such treatment further offering a clinically acceptable safety profile.

### SUMMARY OF THE INVENTION

The present invention provides a method for synergistically treating dyslipidemia in a mammal, comprising administering to a mammal in need thereof, an effective amount of a compound of Formula I:

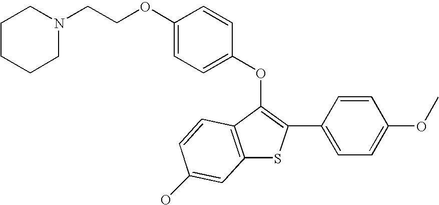

or a pharmaceutically acceptable salt, solvate, or polymorph thereof and at least one statin.

### DETAILED DESCRIPTION

The present invention relates to a method for treating dyslipidemia in a mammal in need thereof. The method of the present invention comprises administering to a mammal in need thereof, a pharmaceutically effective amount of arzoxifene in combination with a statin.

The methods of the present invention are administered to mammals in need of such treatment. Generally, it is preferred that the mammals are human patients. It is generally preferred that the mammals are human patients in need of dyslipidemia treatment. In another embodiment, it may be preferred that the dyslipidemia condition is diagnosed by a qualified health professional. As used herein, the term “dyslipidemia” means a condition in which triglycerides are elevated and/or LDL-C is elevated and/or HDL-C is lower than desired. It may be preferred that the patient in need of treatment has both elevated triglycerides and elevated LDL-C. In another embodiment, the patient in need of treatment wishes to raise their HDL-C level. Additionally, in another embodiment, the patient may wish treatment directed toward primarily lowering LDL-C levels. The term “dyslipidemia” collectively contemplates any undesired lipid profile in a patient.

The term “statin” as used herein means such compounds as simvastatin disclosed in U.S. Pat. No. 4,444,784; pravastatin, disclosed in U.S. Pat. No. 4,346,227; cerivastatin, disclosed in U.S. Pat. No. 5,502,199; mevastatin, disclosed in U.S. Pat. No. 3,983,140; velostatin, disclosed in U.S. Pat. Nos. 4,448,784 and 4,450,171; fluvastatin, disclosed in U.S. Pat. No. 4,739,073; compactin, disclosed in U.S. Pat. No. 4,804,770; lovastatin, disclosed in U.S. Pat. No. 4,231,938; dalvastatin, disclosed in European Patent application Publication No. 363934 A1; atorvastatin, disclosed in U.S. Pat. Nos. 4,681,893 and 5,273,995; dihydrocompactin, disclosed in U.S. Pat. No. 4,450,171; bervastatin, disclosed in U.S. Pat. No. 5,082,859; and rosuvastatin (Crestor™), disclosed in U.S. Pat. No. 6,316,460, 6,589,959, and 6,858618. A preferred statin for co-administration with arzoxifene can be simvastatin. A preferred statin for co-administration with arzoxifene can be atorvastatin. A preferred statin for co-administration with arzoxifene can be pravastatin. The three statins simvastatin, atorvastatin, and pravastatin are well known to the skilled artisan. As used herein the term “low dose” statin means the lowest dosage approved by the US FDA for administration to humans. As used herein the term “low dose atorvastatin” means the lowest dosage or less of atorvastatin that is approved by the FDA for use in humans. As used herein the term “high dose atorvastatin” means greater than the lowest dosage approved by the FDA for use in humans. The term “low dose simvastatin” means the the lowest dosage or less of simvastatin that is approved by the FDA for use in humans. As used herein the term “high dose simvastatin” means greater than the lowest dosage approved by the FDA for use in humans. The term “low dose pravastatin” means the lowest dosage or less of pravastatin that is approved by the FDA for use in humans. As used herein the term “high dose pravastatin” means a dosage that is greater than the lowest dosage approved by the FDA for use in humans.

As used herein, “effective amount” refers to an amount of arzoxifene, or a salt or solvate thereof, capable of treating dyslipidemia, when administered in combination with a statin, in a patient in need thereof. As used herein “synergistic” or “synergistically” mean that the composition or co-administration exerts an effect that is greater than the sum of the individual effects of arzoxifene and statins when administered separately.

As used herein, the term “co-adminstration” means that arzoxifene or Formula I is administered during the same treatment cycle as the statin. Such co-administration includes a dosing regimen in which the arzoxifene or Formula I is administered at the same time as the statin. Such co-administration means that these components can be administered together as a composition or as part of the same, unitary dosage form. “Co-administration” also includes administering arzoxifene or Formula I and a statin separately, but as part of the same therapeutic treatment program or regimen. The components need not be administered at essentially the same time, although they can be administered at the same time, if desired. Thus, co-administration can mean that the statin is administered daily while arzoxifene is administered less than daily using a consistent treatment schedule. The term co-administration may mean that the arzoxifene and statin are administered sequentially as separate unit dosage forms together using the same dosing interval for both arzoxifene and the statin. Such “less than daily” dosing of arzoxifene must be administered using a dosing regimen providing the patient with a continuous effective amount of arzoxifene during the treatment cycle.

The combinations of this invention can be administered in a controlled release formulation. Such controlled release formulations of the combination of this invention may be prepared using methods well known to those skilled in the art. The method of administration will be determined by the attendant physician or other person skilled in the art after an evaluation of the subject's condition and requirements.

The term “diagnose” means that a physician or other health care professional qualified to clinically diagnose the described condition has determined that the subject is in need of treatment for dyslipidemia or a subset of the conditions contemplated by the term “dyslipidemia”.

The term “solvate” refers to an aggregate that comprises one or more molecules of the solute, such as an aggregate of compound I, with one or more molecules of solvent. Suitable solvent molecules are those commonly used in the pharmaceutical art, which are known to be non-detrimental to the recipient, e.g., water and ethanol. The preparation of solvated forms of arzoxifene has been described in the art.

Although the free-base form of arzoxifene can be used in the formulations and methods of the present invention, preferably, the compounds are in the form of a pharmaceutical salt. Thus, the term “pharmaceutically acceptable salt” refers to acid addition salts of compound I which are substantially non-toxic at the doses administered and are commonly known in the pharmaceutical literature. See e.g. Berge, S. M., et al., J. Pharm. Sci., 66(1), (1977). Pharmaceutically acceptable salts of arzoxifene are described in the '474 patent.

The “kit” envisioned by the present invention is for use by a consumer to treat dyslipidemia. It can be desirable when the treatment of dyslipidemia can slow or reverse atherosclerosis in the patient in need of such treatment.

The dosage to be administered may vary depending upon the physical characteristics of the patient, the severity of the patient's symptoms, and the means used to administer the drug. The specific dose for a given patient is usually set by the judgment of the attending physician.

Arzoxifene can be administered with a statin on a daily basis for the treatment of dyslipidemia. A typical daily dose of arzoxifene would contain a nontoxic dosage level of from about 1 mg to about 200 mg/day. Preferred daily doses generally will be from about 1 mg to about 50 mg/day. Preferred daily doses for the treatment dyslipidemia are generally from about 5 mg to about 20 mg/day. A dosage of 20 mg/day arzoxifene may be preferred for some dyslipidemia patients. Such a dosage may be given as a single dose or may be divided into two or three separate doses per day as appropriate.

Azoxifene may be effective when administered less than daily for the treatment of dyslipidemia in combination with a statin, which statin may be administered daily while the arzoxifene dosage is administered less than daily. Such less than daily dosing includes, for example, administering arzoxifene every other day, every third day, once per week, or every other week in combination with a statin, which statin may be administered daily or less than daily.

### EXAMPLES

The following examples further describe and demonstrate embodiments within the scope of the present invention. The examples are given solely for the purpose of illustration and are not to be construed as limitations of the present invention as many variations thereof are possible without departing from the spirit and scope of the present invention.

Clinical Studies

These studies are conducted as complete balanced factorial design with different statins.

Atorvastatin

Studies are conducted using all doses of atorvastatin (10 mg, 20 mg, 40 mg and 80 mg), arzoxifene and placebo. This study is designed to include 10 cells with 37 patients per cell. A total of 370 patients are included in the study.

This study may be conducted without the placebo-placebo cell resulting in total enrollment of 333 patients.

This study may further be conducted using only the high and low dose of atrovastatin. This study design requires only 6 cells with 37 patients per cell for a total of 222 patients per study.

Simvastatin

Studies are conducted using a high and a low dose of simvastatin, arzoxifene, and placebo. This study is designed to include 10 cells with 37 patients per cell.

Alternatively, the study can be conducted using 5/6 cells.

Pravastatin

Studies are conducted using a high and a low dose of pravastatin, arzoxifene, and placebo. The full free factorial study is designed to include 8 cells with 37 patients per cell.

This study can also be conducted using only a high and low dose of pravastatin using 5/6 cells.

Patients' total cholesterol, LDL-C, ApoB, and HDL-C are measured at baseline using accepted assays. Patients are monitored after one month, three months and six months to assess treatment effect on patients' dyslipidemia.

Biomarkers may be useful in assessing risk of developing a disease, establishing the diagnosis of the disease, predicting or monitoring the course of a disease, For some biomarkers or combinations of biomarkers, baseline values correlate with subsequent disease progression, whereas for others, the biomarker expression over time or change over time shows better correlation with radiographic outcomes. Inter-individual variability hampers the application of these biomarkers to the assessment and prognostication of particular subjects or patients, e.g., for the purpose of selecting subjects for trials of treatments intended to alter disease progression. The newer biomarker assays, may minimize the variability and enhance the predictive power of biomarkers for cardiovascular disease progression. These biomarkers are attractive tools for early phase clinical studies.

### Clinical Study

A study to demonstrate the clinical effects of arzoxifene on the treatment and/or progression of dyslipidemia is designed as follows. The study is a 6-month, multicenter, Phase IIa, randomized, double-blind, parallel, placebo-controlled trial in postmenopausal women with documented dyslipidemia. The study population will be ≧2 years postmenopausal.

Screening safety laboratory studies and baseline total cholesterol, LDL-C, ApoB, and HDL-C will be performed. Only subjects who meet all eligibility criteria after completion of these tests will be asked to return for randomization to study treatment. At visit 2, eligible subjects, will be enrolled and randomized to receive low dose arzoxifene plus high dose simvastatin, low dose arzoxifene plus low dose simvastatin, low dose arzoxifene plus placebo, low dose simvastatin plus placebo, high dose arzoxifene plus placebo, high dose simvastain plus placebo or placebo:placebo during the double-blind treatment phase. Subjects will continue taking the blinded drug treatment assignment throughout the remainder of the study.

### Treatment Phase

Subjects will receive double-blind study medication for 26 weeks throughout the treatment phase. In another, more preferred design of this study, subjects will receive double-blind study medication for 12 weeks throughout the treatment phase. Biomarkers will be assessed at baseline and after 12and, in studies in which patients are dosed for 26 weeks, at 26±2 weeks of study treatment. Biomarker specimens will also be collected after 3±1 and 6±1 weeks of study treatment.

### Study Population

Study subjects are women at least 2 years postmenopausal AND between 50 and 70 years old, inclusive. At study entry, women must have clinically elevated cholesterol levels.

In clinical use, the specific doses of arzoxifene will, of course, be determined by the particular circumstances surrounding the case. Similarly, the route of administration is a factor determined by the specifics of each case. Thus, the exact dose and route of administration are best determined by the attending physician.

We claim:

1. A method for treating dyslipidemia in a mammal, comprising co-administering to a mammal in need thereof, an effective amount of a compound of Formula I: 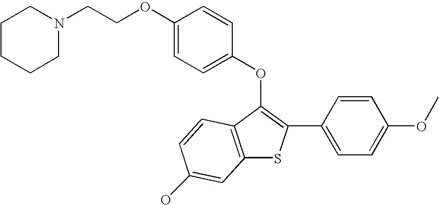
or a pharmaceutically acceptable salt or solvate thereof; and at least one statin.

2. A method as claimed by claim 1 wherein the effective amount of arzoxifene and effective amount of the statin synergistically treats dyslipidemia.

3. A method as claimed by claim 2 wherein the mammal is a human patient.

4. (canceled)

5. A method as claimed by claim 3 wherein the human patient is a man.

6. A method as claimed by claim 3 wherein the human patient has been clinically diagnosed as being in need of treatment for dyslipidemia.

7. A method as claimed by claim 3 wherein the dyslipidemia is hyperlipidemia.

8. A method as claimed by claim 7 wherein the dyslipidemia is elevated triglycerides.

9. A method as claimed by claim 7 wherein the dyslipidemia is elevated LDL-C.

10. (canceled)

11. (canceled)

12. A method as claimed by claim 7 wherein the statin is selected from the group consisting of atorvastatin, simvastatin, and pravastatin.

13. (canceled)

14. (canceled)

15. (canceled)

16. (canceled)

17. (canceled)

18. (canceled)

19. A method as claimed by claim 12 wherein Formula I effective amount is 10 mg.

20. A method as claimed by claim 12 wherein Formula I effective amount is 20 mg.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. A method as claimed by claim 1 wherein the dosing regimen for the compound of Formula I is once-weekly dosing.

36. (canceled)

37. (canceled)

38. A method for treating dyslipidemia in a mammal in need thereof comprising administering to said mammal, a pharmaceutically effective amount of arzoxifene, and a statin, as a unit dosage according to a continuous schedule having a dosing interval selected from the group consisting of once-weekly dosing and twice-weekly dosing.

39. A method as claimed by claim 38 wherein the pharmaceutically effective amount of arzoxifene and statin provides a synergistic treatment effect.

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. A kit for treating dyslipidemia in a patient in need thereof comprising at least one pharmaceutically effective unit dosage of arzoxifene and at least one statin for administration according to a continuous schedule having a dosing interval selected from the group consisting of once-daily, once-weekly dosing and twice-weekly dosing.

57. (canceled)

58. (canceled)

59. (canceled)

60. (canceled)

61. (canceled)

62. A composition comprising a pharmaceutically effective amount of arzoxifene, and a pharmaceutically effective amount of at least one statin, and a pharmaceutically acceptable excipient.

63. A composition as claimed by claim 62 wherein the composition is useful for the treatment of dyslipidemia.

64. A composition as claimed by any claim 63 wherein wherein the statin is selected from the group consisting of atorvastatin, simvastatin, and pravastatin.

65. (canceled)

66. (canceled)

67. (canceled)

68. A composition as claimed by claim 64 wherein the composition is a unit dosage.

69. A composition as claimed by claim 68 wherein the pharmaceutically effective amount of arzoxifene is from 5 mg to 50 mg.

70. (canceled)

71. A composition as claimed by claim 64 wherein the pharmacological effect of arzoxifene and the statin is synergistic.
